(19) United States
(12) Patent Application Publication
(10) Pub. No.: US 2020/0199505 A1
(43) Pub. Date: Jun. 25, 2020

(54) PRODUCTION PROCESS FOR HIGH PURITY ALGAE

(71) Applicant: JIANWEI CHEN, Calgary (CA)

(72) Inventor: JIANWEI CHEN, Calgary (CA)

(21) Appl. No.: 16/620,750

(22) PCT Filed: Jun. 8, 2018
(86) PCT No.: PCT/CA2018/050691

§ 371 Date: Dec. 9, 2019

Related U.S. Application Data
(60) Provisional application No. 62/517,802, filed on Jun. 9, 2017.

(51) Int. Cl. C12M 1/00; C12M 1/12; C12N 1/12

(52) U.S. Cl. CPC C12M 21/02; C12M 27/20; A23L 3/0155; C12M 23/04; C12N 1/12; C12M 31/10

(57) ABSTRACT

A photo-bioreactor for growing a photosynthetic culture comprising: a channel through the bioreactor having an inlet and an outlet providing a labyrinth path from the inlet to the outlet, configured to contain the photosynthetic culture, wherein the channel is formed by a plurality of ribs; and a light emitting diode panel along a side wall of the photo-bioreactor. A method of product preservation, comprising: treating a packaged product with a first cycle of high pressure processing; incubating the packaged product allowing spore germination; and treating the packaged product with a second cycle of high pressure processing.

### FIELD OF INVENTION

This invention relates to a photo-bioreactor device for growing a photosynthetic culture and a method of preserving a final packaged product.

### BACKGROUND

Algae is commercially cultivated for a variety of uses including fuel production and bioplastic manufacture. A typical commercial algae production process uses an open pond and solar light. This process may be a cost effective way for growing algae. However, if a more pure final product is desired, a potential problem encountered with the open production of algae is contamination. Contaminants in air and water such as dust, heavy metals, chemical residues, and biological contamination of bacteria or foreign algae may be a problem. Another problem with open pond cultivation is that it may require a significant volume of water. The water supply in such a process is typically a river or a lake, which may additionally contaminate the algae product.

Other commercial uses of algae include food consumption, cosmetic applications and pharmaceutical applications. These applications may have more stringent product purity requirements such that it may not be possible to use an open pond cultivation. In these instances, algae can be cultivated indoors. Indoor bioreactors may be closed or semi-closed to prevent water or air contamination. Indoor bioreactors that may provide a suitable aqueous environment for healthy algal growth may include the following components: water, nutrient sources, carbon dioxide, and light. In locations with sufficient sunlight, solar light may be used as the light source. However, solar light may not suitable in locations where the sunlight is insufficient or inconsistent such as at high-latitudes.

In addition, if a system is intended for large-scale production of algae, then solar illumination may also require a shallow bioreactor vessel that spreads the algae culture over a large surface area so that algae cells do not shade one another. This can be very costly as a large area of land occupation may be required. Further disadvantages of solar illuminated algal bioreactors may include:
temperature control challenges;
mechanical challenges of orienting the bioreactor to an appropriate sunlight capture angle (this angle would likely change during the day and throughout the year); and
sensitivity challenges, as some algal species may not grow in direct solar light.

The purity of the algae may also be affected by the packaging. Untreated, harvested algae has a very short shelf life and may spoil rapidly due to the degradation of algae and the growth of undesirable foreign algae as well as growth of bacteria.

### SUMMARY OF THE INVENTION

A photo-bioreactor for growing a photosynthetic culture in an aqueous liquid and harvesting a photosynthetic culture is provided, in an embodiment, the photo-bioreactor comprising: a channel through the bioreactor having an inlet; an outlet providing a labyrinth path from the inlet to the outlet, configured to contain the photosynthetic culture, wherein the channel is formed by a plurality of ribs; and a light emitting diode panel placed along a side wall of the photo-bioreactor.

A method of packaged product preservation is also provided, in an embodiment comprising: treating a packaged product with a first cycle of high pressure processing; incubating the packaged product; and treating the packaged product with a second cycle of high pressure processing.

### BRIEF DESCRIPTION OF THE DRAWINGS

FIG. 1 is a schematic flow chart of the photo-bioreactor.

FIG. 2 is a side view of the buffering tank.

FIG. 3 is a side, front, and top view of the photo-bioreactor.

FIG. 4 is an exploded, perspective view of the LED panel.

### DETAILED DESCRIPTION

The detailed description and examples set forth below are intended as a description of various embodiments of the present invention and are not intended to represent the only embodiments contemplated by the inventor. The detailed description includes specific details for the purpose of providing a comprehensive understanding of the present invention. However, it will be apparent to those skilled in the art that the present invention may be practiced without these specific details.

Cultivation of algae by an indoor bioreactor may also be achieved by using an artificial light source. These systems may provide stable illumination and stable temperature conditions that would likely promote algal growth. Stable conditions can also provide consistent production and may result in a high purity of a desired algae species with less contamination.

The present invention is a closed bioreactor using a light-emitting diode (LED) panel as the light source.

The broadest embodiment of the present invention teaches a photo-bioreactor for growing a photosynthetic culture of liquid solution and algae, the bioreactor comprising: an inlet; an outlet; a side wall; a channel from the inlet to the outlet having a labyrinth path configured to contain the photosynthetic culture, wherein the channel is formed by a plurality of ribs projecting into the interior of the bioreactor, the ribs create a tubular flow; and a LED panel along the side wall of the photo-bioreactor. The photo-bioreactor maybe be shaped as a panel with side walls that are planar, flat and defined between side edges, a bottom edge and a top edge. There is a front side wall and a rear side wall together giving the bioreactor a thickness from side-to-side walls The thickness is much less than the side edge, the bottom edge or the top edge length and width dimensions.

The photo-bioreactor of the present invention may be oriented vertically. In this orientation, the frame carries the weight of the aqueous solution and avoids the weight being carried over to a large unsupported area. In addition, this orientation facilitates the use of thin, light, transparent, plastic sheets to define the side walls of the photo-bioreactor and reduces light transmission losses of the photo-bioreactor. The ribs provide a labyrinthine fluid flow path and constrain the side walls to prevent ballooning under high fluid pressure inside the flow path of the bioreactor. The vertical orientation of the photo-bioreactor allows any gas bubbles to be directed to a central region of the photo-bioreactor further reducing light losses. The vertical orientation of the photo-bioreactor may also help prevent fluid leaks, or at least leaks can be contained in a small footprint.

Plastic panel sheets are used to construct the photo-bioreactor. One example of plastic sheets is a commercially available triple-wall hollow sheet. These sheets typically measure 25′ in length, 4′-7′ in width and are thin. Example sheet material thicknesses (sometimes referred to as the “skin thickness”) may be 3 mm for an acrylic panel sheet or less than 1 mm for a polycarbonate panel sheet. The total thickness of the assembled photo-bioreactor may depend on the available space where the photo-bioreactor will be placed and the dimensions of the LED panel. For example, a sheet with dimensions of 4′ by 8′ with assembled thickness of 6 to 32 mm, may be compatible with 4 pieces of a 2′ by 4′ LED panel that may fully cover the side wall of the photo-bioreactor.

The plastic panel sheets may be comprised of glass or plastic materials such as transparent polycarbonate, polyethylene or acrylic. The plastic affords a high light transmitting efficiency (90-95% visual light for acrylic vs 80-88% for polycarbonate with thicker panels absorbing more light and having lower light transmission efficiency) and the polycarbonate sheet is a cost effective and high impact strength material. The choice of plastic depends on the desired application. For example, in the production of human consumable algae, acrylic would likely be used, as polycarbonate may release a toxic chemical compound, such as bisphenol A (BPA).

The bioreactors have two openings an inlet 111 and an outlet 113 with the channel in between. Each channel extends from an inlet, which is generally on a first side edge of the bioreactor and the channel extends toward the other side edge and then curves around 180° and runs back toward the first side edge. The channel is tortuous, having a number of these side-to-side lengths called runs, and curves in between. The channels side-to-side runs may be equidistant from one another and are about 8-60 mm apart. The channels are the roughly parallelogram-shaped cross-sectional spaces defined between the side walls and adjacent pairs of ribs. The channels may be defined within the bioreactor by casting of panel sheets with integral ribs, milling of flat sheets to have ribs, or mechanical fastening, welding, or gluing of ribs to the inner side(s) of plastic sheets that are to become side walls.

The channel extends in a plane substantially parallel with the side wall planar exterior surface. As such, the channel is always placed only a short distance from the outer surface of the sidewall, the channel has a substantially consistent depth.

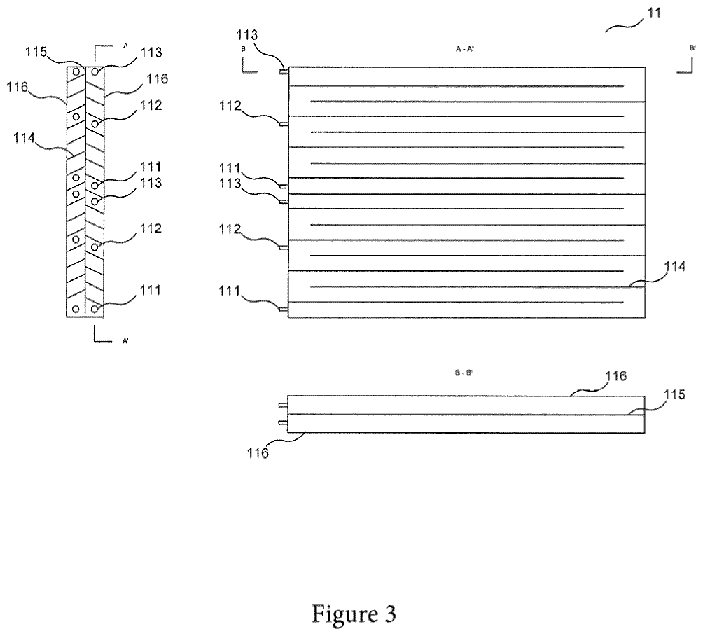

The channel side-to-side runs may configure the channel as a labyrinth. A plurality of ribs form the labyrinth channels. The spacing between the ribs provides stable flow. In a preferred configuration, as shown in FIG. 3, the photo-bioreactor includes a middle wall between two side walls, with one set of ribs spanning from the first sidewall to the middle wall and a second set of ribs spanning from the second sidewall to the middle wall. The ribs may be angled from the connection to the side wall upwards towards the panel upper edge and the middle wall, as shown in FIG. 3 to form a head space, for example an upper corner within the channel along its side to side runs. The angle (i.e.

the upper corner) allows gas bubbles to rise to the top-back (when considering the outer side wall as the front) or when a middle wall is included the top-central corner of the channel which is consistently the furthest from the side wall outer surface and thereby, furthest from the LED light. This may prevent bubbles from getting trapped in the channel as bubbles that may be trapped can hinder the photosynthetic culture from obtaining direct light exposure. The photo-bioreactor is made up of a plurality of sections.

The overall length of a channel between inlet and outlet in one section may be between 80-120 m. This distance prevents over-saturation of oxygen produced by algae and limits oxygen inhibition in the culture. Longer distance channels with continuous oxygen generation by algal photosynthesis may result in a high dissolved oxygen concentrations in the aqueous solution, for example higher than 300% air saturation concentrations that can inhibit algal growth. Each photo-bioreactor may have inlet 111 at the bottom, a gas inlet 112 in the middle and liquid outlet at the top 113.

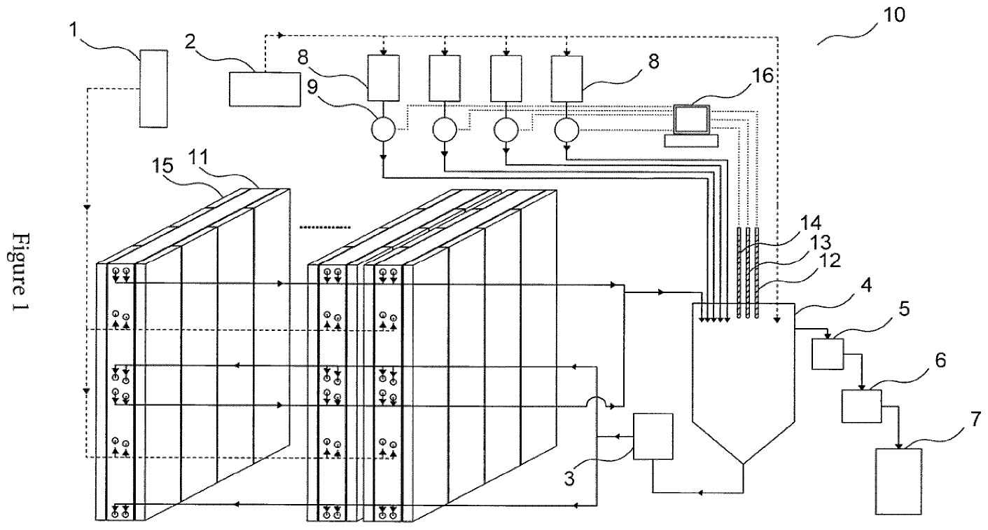

The photo-bioreactor may include a plurality of sections, each having the parts noted herein before. The photosynthetic culture coming from a supply, such as a buffering tank may flow into the multiple sections of the photo-bioreactor that are connected in parallel. The sections are connected together in parallel and are stacked one on top of the other. For example, FIG. 3 shows two sections stacked vertically; and FIG. 1 shows two sections stacked vertically and multiple sections stacked horizontally.

Controlled mixing in the present invention may be achieved by gentle fluid mixing caused by the fluid flow direction changes within the labyrinth path of the channel, whereby horizontal tubular flow along the side wall changes to vertical tubular flow along the edge wall then curves back to another side-to-side run. Controlled, but gentle, mixing of the photosynthetic culture is important to provide uniform light absorption to the photosynthetic culture and to provide a good distribution of nutrients to the algae cells.

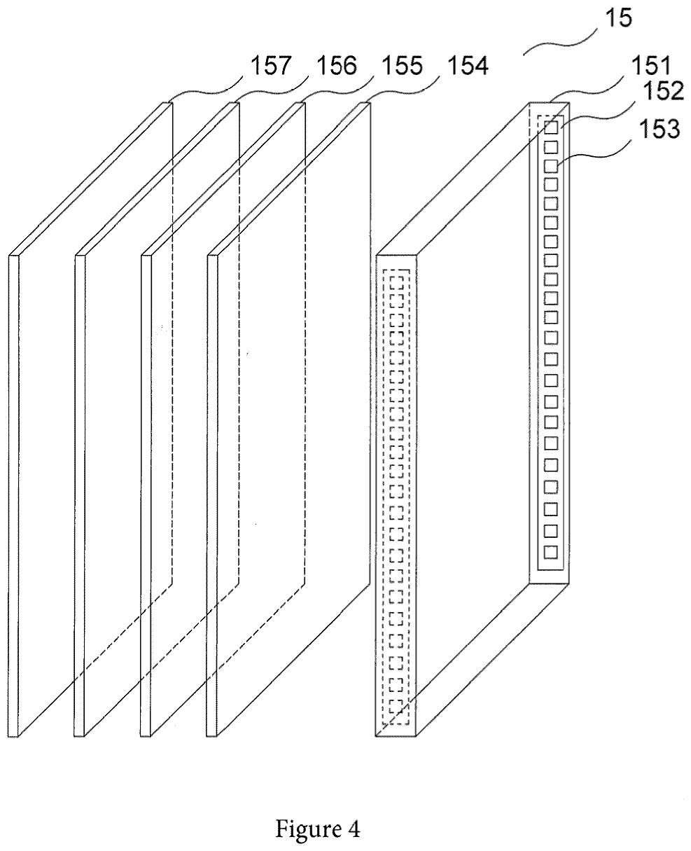

In one embodiment, a LED panel may be used as the artificial light source for the bioreactor. The key components of the LED panel are a metal frame, a white LED strip, a light transmitting layer and a power driver. The LED strips are mounted onto at least two inner edges of the metal frame, with the LED bulbs facing inward as shown in FIG. 4. The light transmitting layers may include: a diffusion plate 154, a light guide plate 155, a reflective paper 156 and a back plate 157. The combination of these layers allows the light to diffuse from the side wall to the entire photo-bioreactor providing a uniform illumination. A power driver provides a constant electric current to the LED panel using a residential or other conventional AC power supply. These light fixtures are commonly used in offices, schools or large kitchens to provide a uniform non-glaring light. One example of an LED panel that may be used in the present invention and is a commercially available, edge lit flat, rectangular panel with the following dimensions: a 2′ by 4′ height; and a 8-30 mm thickness. Depending on the constituting material of the plates 154, 155 the light transmitting efficiency of the LED panel may be in the range of 80-90%.

The power uptake for a LED panel may be between 36-108 W, with a photon density of about 100-300 μmol/m²s. The low photon density coupled with an even photon distribution in the present invention may reduce the photo-inhibition and as such may be optimal for efficient photon capture by the algae.

The photo-bioreactor may have multiple LED panels adhered to the side walls and fully cover the side walls of the photo-bioreactor. The majority of photons emitted from the LED may pass into the channels from the side walls and some photons may travel through the ribs.

Algae cultivation by LED panels may be advantageous over sunlight cultivation as it may allow control of the wavelength of emitted light, such LEDs are known as monochromatic LEDs and can increase the algal growth in comparison to natural sunlight. In addition, a monochromatic LED may selectively promote growth of a specific target algae species. In another embodiment, the white LED bulbs may be substituted with one or more types of monochromatic LEDs that may emit light in a narrow wavelength range. The choice of the specific wavelength range depends on the target algal species' maximum light absorption. For example, Arthrospira platensis has maximum absorption at a wavelength of around 440 nm, 620 nm and 680 nm, therefore a combination of monochromatic LED panels emitting light at these three wavelengths may be mounted onto the side wall of the photo-bioreactor, thus increasing growth of Arthrospira platensis.

An advantage of the present invention is that it provides a large surface area of algae growth with a short light path, which is through the side wall plastic panel sheets and then directly into culture in the channels, which have a very small depth (horizontal) from side wall to middle wall. Light absorption may be constrained by the self-shading effect of algal cells and therefore light may not be able to penetrate a depth beyond only a few centimeters. The short light path of the present invention along with very high algal concentrations provides effective culture of the aqueous solution. This allows reduced water use that has significant economic and surface area benefits. Another advantage of the present invention may be that a high maximum amount of light delivery to the photosynthetic culture is achieved compared to sunlight because the LED panel may be in direct contact with the photo-bioreactor. Another advantage of direct contact to the LED panel is that the photo-bioreactor may dissipate the heat produced by the LED panel to the photosynthetic culture. This is advantageous because the preferred temperature for algal growth is between the range of 30-40° C. As such, the heat from the LED panel can be used to raise the temperature of the photosynthetic culture without additional heating being required.

In another embodiment, the present invention provides in situ tailoring of input sources to the photo-bioreactor that may be required to harvest a specific species of algae. One example of an input source is gas, such as nitrogen. These gases are introduced into the system. For example, a nitrogen generator (generating 95-99% pure nitrogen) or a nitrogen gas cylinder may be attached to the photo-bioreactor 1. Nitrogen is introduced to the photo-bioreactor at a rate of about 10-50% of the liquid flow rate. Nitrogen is used to dilute the produced oxygen in the photo-bioreactor so that the oxygen partial pressure is low in the head space, located in the upper cavity between the ribs and the middle wall of the photo-bioreactor. In addition, nitrogen may help stabilize the dissolved oxygen concentration in the photosynthetic culture, because high dissolved oxygen concentrations may be toxic to algae. Nitrogen also creates bubbles in the photosynthetic culture providing better mixing. Nitrogen may help break up biofilms that adhere to the inner walls of the photo-bioreactor.

The photo-bioreactor may have additional components, (see FIG. 1) such as:
a) a vessel where the photosynthetic culture is first introduced, referred to as a buffering tank 4;
b) an air compressor 2 to provide air flow into the buffering tank;
c) sensors in the buffering tank for detecting changes in temperature 14, oxygen 13, and pH 12;
d) a pump 3 such as a diaphragm pump or a membrane pump to cycle the photosynthetic culture from the buffering tank to the photo-bioreactor;
e) a nutrient source, referred to as a concentrated nutrient stock 8;
f) a computer 16 controlling the nutrient sources; and
g) a filtration device to remove the aqueous liquid from the harvesting culture before packaging 5, referred to as a vibration screener.

Some of these known components are briefly described below.

Buffering Tank

The buffering tank is a semi-closed cone. The buffering tank has many functions, such as: preparing the photosynthetic culture for growth within the photo-bioreactor, serving as a backflow vessel for harvesting culture, and holding the harvesting culture before it is pre-packaged. There may be constant air bubbling in the buffering tank in order to create a homogeneous mixture and to reduce the dissolved oxygen concentration. A continuous flow of photosynthetic culture may be introduced into the buffering tank, while at the same time the diaphragm pump, may pump the photosynthetic culture into the photo-bioreactor, this forms a continuous loop.

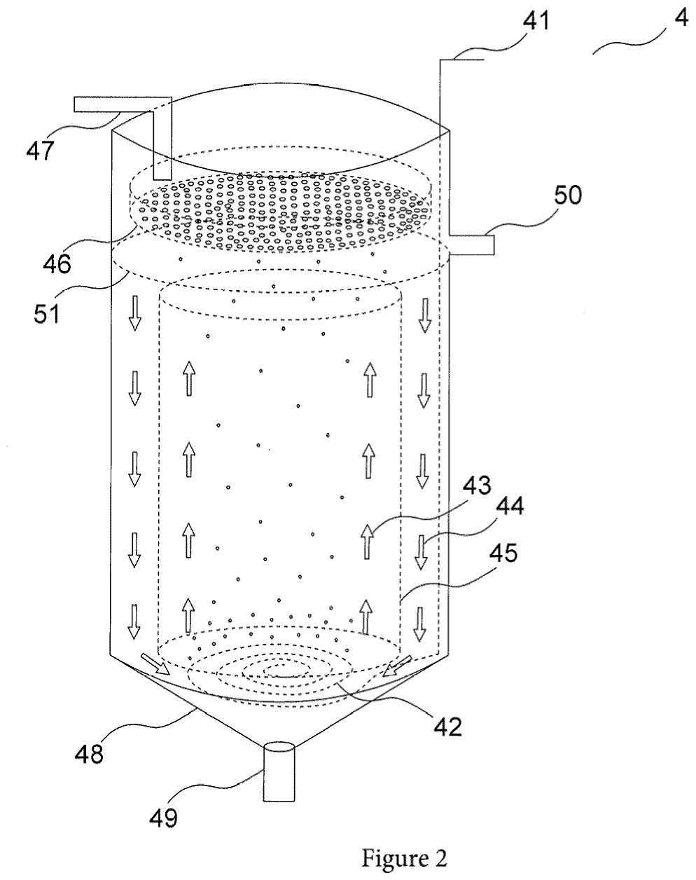

The buffering tank, as shown in FIG. 2 may be comprised of: a conical base 48, a baffle wall 45, a screen, a gas line, and a cover. The conical base may be comprised of stainless steel or a high quality plastic. The working capacity of the buffering tank may be between 10-50% of total liquid volume. The height to diameter ratio of the buffering tank may exceed two, with higher ratios giving better mixing performance. The conical base may be angled at a 30° to 60° angle relative to the horizontal. The buffering tank may have openings, such as: a bottom outflow outlet 49, an overflow outlet 50, and a fluid return line 47. The baffle wall of the buffering tank may contain a cylindrical ring comprised of the same material as the buffering tank. The baffle wall may have a base diameter of about 80% to 90% of the conical base of the buffering tank. The base of the baffle wall is 0.1 m to 0.2 m higher than the top opening of conical base of the buffering tank. There may be a screen, which acts as a sieve, above the baffle wall, for example about 0.2 m to 0.6 m above the baffle wall. The sieve may be made of same material as the baffle wall. The diameter of the sieve may be 95-98% that of the conical base. A plurality of holes may be evenly spread across the sieve with a diameter of about 2 mm to 5 mm. The sieve may be used to distribute the backflow harvesting culture from the photo-bioreactor evenly into small droplets before mixing with the bulk harvesting culture. The small droplets may exchange dissolved gas with air due to the large surface area. The small droplets may reduce the foam floating on top of the bulk harvesting culture. Another component of the buffering tank is an inlet gas line. The gas line may be made of stainless steel or a high quality plastic, with an outside diameter of ¼″ to ½″. The gas line may come from the top of the buffering tank to the base of the tank. The gas line may have a helix structure, with a plurality of holes in the line in order to release air. A cover may be placed on top of the buffering tank to inhibit air pollution contamination.

Air Compressor

An air compressor may be used to provide air flow into the buffering tank and to various other components connected to the buffering tank. The air compressor also allows for a fine particle filtration to remove any contaminant present in the air of the buffering tank.

Pump

A diaphragm or membrane pump, may be used to transport the photosynthetic culture from the buffering tank to the photo-bioreactor and eventually back to the buffering tank. The diaphragm pump may be used as it does not create a high shear force and minimizes forces applied to the photosynthetic culture.

Vibration Screener

A vibration screener may be used to separate the aqueous liquids from the harvesting culture. The screen hole size may depend on the target algae species. After travelling through the vibration screener, the harvesting culture is washed and packaged.

Concentrated Nutrient Stock

A concentrated nutrient stock may have separated flow streams for different nutrition stocks controlled by an independent peristaltic pump. This can prevent precipitation of the nutrient stock and inhibit the formation of a biofilm on the surface of the bulk culture. The concentrated nutrient stock separately pumps each individual nutrient to the buffering tank. Separate vessels for each nutrient stock may also provide a high shelf for each individual nutrient stock. Once the nutrient is introduced into the buffering tank, it may be diluted, which can prevent precipitate formation. The concentrated nutrient stock allows for real-time addition of nutrient sources to the buffering tank with any adjustments being controlled by a computer 16.

The concentrated nutrient stock comprises between 3 to 5 independent tanks and this may be adjusted or tailored to the specific algae species. Table 1 is an example of the potential nutrient stocks that may be added to the harvesting culture in the buffering tank.

TABLE 1
Some examples of the concentrated nutrient stocks

| | Medium | | | | |
|---|---|---|---|---|---|
| | | | | | Trace |
| | Stock I | Stock II | Stock III | Stock IV | elements |
| Chemicals | NaHCO₃; | KH₂P O₄; | MgSO₄; | FeSO₄; | Na₂EDTA |
| | Na₂CO₃; | | CaCl₂; | Na₂EDTA | CuSO₄; |
| | | | KCl; | | ZnSO₄; |
| | | | NaCl; | | CoCl₂; |
| | | | KNO₃; | | MnCl₂; |
| | | | | | Na₂MoO₄; |
| | | | | | H₂SeO₃; |
| | | | | | NaVO₄; |

Another broad aspect of the present invention, teaches a method of product preservation, comprising: treating a packaged product with a first cycle of high pressure processing; incubating the product; and treating the packaged product with a second cycle of high pressure processing.

The final product is treated with a first cycle of High Pressure Processing (HPP), a non-thermal process that sterilizes sealed product by applying ultra-high pressure uniformly to the packaged product. The process may help to extend the storage time of a packaged product. As a prerequisite, the product should have a high water content and be pre-packaged in a flexible container, for example a plastic pouch. The HPP comprises applying pressure of up to 600 MPa for a specified period of time, such as 5-8 minutes, in a closed vessel containing a packaged product at ambient or other temperatures. The HPP method does not typically form or break covalent bonds of a small molecule, but may instead change bond angles and exchange disulfide bonds. Some enzymes may be denatured from the treatment, but there is likely no change in the chemical composition.

One cycle may not be enough as there may be some microbial spores that may be too resistant to be killed or denatured after one cycle. One method of preventing spore germination is to maintain the final product pH below 4.6. However, this may not be appropriate for the preservation of a pH sensitive product.

The present disclosure may address this problem by applying a method that incorporates at least two cycles of HPP treatment. The two cycles are substantially identical in this procedure. The two cycles are separated by an incubation time allowing for the germination of spores to take place. For example, after running one cycle of HPP, the product is left at ambient temperature and pressure for about 1 to 48 hours and then is treated to a second HPP cycle. The advantage of this methodology is that after one cycle of HPP, most organisms are deactivated. The remaining microbial spores that are resistant to the first cycle are grown for a specific time under ambient conditions. Once germinated, they may be deactivated by a second HPP cycle. As a result, the final packaged product is high in purity and may have a high self-life. A further refinement of this two-cycle HPP treatment is to initially sample the product to determine the specific species of spores present and optionally measure the pH. Different species may require differing spore germination times and may be pH sensitive. Therefore, the ambient temperature and pressure dwell time may be selected based on the known spore species.

Method of Producing High Purity Algae

Once the system is assembled, it is important to first run a disinfection cycle prior to inoculating the photosynthetic culture. The buffering tank may be filled with a non-toxic disinfection solution, such as a NaCl or a citrate acid solutions in varying concentrations such as in 20% in solution. The disinfection solution may be kept in the buffering tank for at least two hours. After at least two hours, the disinfection solution may be drained from the system and the system may be rinsed with clean water.

Once the system has been cleaned, the photosynthetic culture is introduced into the buffering tank. The outflow outlet of the buffering tank 49 supplies the photo-bioreactor with the photosynthetic culture, rich in dissolved oxygen (DO) with a concentration of 100% to 200% of air saturation. The dilution ratio at this stage may be within 5-10% of the original inoculation solution with fresh media. Therefore, a minimum of 10 L photosynthetic culture may be prepared, so that it can mix with up to 190 L of aqueous liquid (1× concentration) in the buffering tank. At this stage, only one section of the photo-bioreactor may be used for culturing, while the others are kept closed to the main line. The LED panel corresponding to the open photo-bioreactor is turned on and a diaphragm pump is operated at a low speed. The multi-channel supply manifold is turned off at this time.

The diaphragm pump delivers the photosynthetic culture in the main line from the buffering tank to the photo-bioreactors that are connected in parallel by a manifold.

When the biomass density reaches up to 1 g dry weight/L, fresh photosynthetic culture (1× concentration) of up to 3.8 cubic meters may be added to the buffering tank. At this point, multiple photo-bioreactors may be open to the main line from the buffering tank as there may be sufficient photosynthetic culture in the buffering tank to fill all sections of the photo-bioreactors. The photosynthetic culture travels from the buffering tank to the photo-bioreactors and back to the buffering tank, this is referred to as recycling.

Once the photosynthetic culture enters the photo-bioreactor it absorbs light emitted by the LED panels as it passes through the channels, and photosynthesis takes place. The LED panels are located along the side walls of the photo-bioreactor. In one configuration, there are multiple LED panels on each side wall of the photo-bioreactor, as shown in FIG. 1 that depicts four LED panels on each side. There may be non-illuminated gaps between the LED panels which correspond to the panel metal frames. Therefore, the photosynthetic culture may experience a time of light exposure and a short time without light as the photosynthetic culture travels along the photo-bioreactor channel adjacent the LED panel frame. This may help the algae to balance a dark reaction and a light reaction.

The photosynthetic culture may produce oxygen gas while travelling through the channels. The produced gas may be transported by the liquid flow through the labyrinth channel, and may gradually form bubbles that congregate at the top of the channel. Bubbles may be visible after the photosynthetic culture has passed through one or two times, and may become large enough to create a mixing within the fluid flow. At this point, the photosynthetic culture may be oversaturated with oxygen gas of up to 300-500% air saturation. As a result, nitrogen gas (95-99% purity) may be introduced into the photo-bioreactor to lower the dissolved oxygen (DO) concentration to as low as 100-200% air saturation, depending on the oxygen to nitrogen ratio. The amount of nitrogen gas that may be added can vary from 10% to 50% of the flow volume. This allows photosynthesis to be continuous, photosynthesis may be inhibited if DO concentrations reach 300% air saturation. After the photosynthesis has taken place in the photo-bioreactor, the medium exits from the outlet 113 of the photo-bioreactor to the main tubing, this allows the culture to flow back into the buffering tank. There may be multiple such cycles until stable production is achieved.

Once the buffering tank is full, the bulk harvesting culture inside the baffle wall may rise and spill over into the void between the baffle wall and the buffering tank. At this stage in the process, dissolved oxygen may be reduced to a level as low as 100% of air saturation. Bubbles may be released into the atmosphere at the top of the buffering tank. During this process, there may be foam on the surface of the harvesting culture.

The backflow from the photo-bioreactor may be sprayed onto a sieve 46 creating small droplets that drop down onto the bulk harvesting culture into the buffering tank, thereby reducing the foam that may be present on the surface. This process may also release dissolved oxygen from the harvesting culture.

When the harvesting culture enters the buffering tank, only dark reactions and respiration of the algae may take place because the buffering tank is not exposed to light. The overall retention time of the harvesting culture in the buffering tank may be 10-40 min, depending on the pump rate and the size of the buffering tank.

When the harvesting culture density reaches 2 g dry weight/L, the concentrated nutrient stock 8 may be turned on. Different nutrients from the concentrated nutrient stock vessels are pumped into the buffering tank continuously, at pre-set pump rates while the pH, oxygen and temperature are monitored by the computer 16. There may be large pH, oxygen or temperature fluctuations at the beginning of the process that may last for days or weeks: the pH may increase from pH 8 to pH 10; the oxygen may reach 300-500% and may need to be lowered by bubbling; the temperature may increase to 3-7° C. above room temperature. However, when the oxygen, temperature and pH parameters are stabilized, this can be an indicator that the photo-bioreactor has achieved a stable production.

At this stage, the harvesting culture is introduced to the vibration screener 5, where the harvesting culture is filtered by particle size screens to separate the culture from the aqueous liquid. The collected culture may be washed with tap water and re-filtered. This washing step may be repeated if necessary. The culture is then introduced to the packaging machine 6, to form a sealed product, such as in a 100mL pouch.

The pouches may be treated to a first cycle of HPP. A HPP cycle may be run at 400-600 MPa for 5-8min at ambient temperature. After the first treatment of HPP, the product may be kept at room temperature for 1-48 hours to allow incubation, specifically for spore germination. The pH of the product is unchanged and may be greater than a pH 4.6. The product is then treated with a second cycle of HPP, using substantially the same parameters as the first cycle. The product formed after the second HPP cycle may be the final packaged product in high purity. The pH of the final product would likely be neutral. If the pH of the product is below 4.6, then spore germination may be inhibited and a second HPP cycle may not be necessary.

A single cycle of HPP may provide 2-8 weeks of final product shelf life, depending on the species of algae.

Using this method, two cycles of HPP may provide in the range of 6-10 months of additional final product shelf life.

### DETAILED DESCRIPTION OF THE FIGURES

FIG. 1.

1. Nitrogen generator

2. Air compressor

3. Diaphragm pump

4. Buffering tank

5. Vibration screener

6. Packaging machine

7. High pressure processing machine

8. Concentrated nutrient stock

9. Quantitative pump

10. System

11. Photo-bioreactor

12. pH sensor

13. oxygen sensor

14. Temperature sensor

15. LED panel light

16. computer.

dash line gas flow, solid line liquid flow, dot line, electric wire

FIG. 2.

4. Buffering tank

41. Compressed air inlet

42. Bubble generator

43. Fluid convection

44. Fluid convection

45. Baffle wall

46. Sieve

47. Flow return line

48. Conical base

49. Outflow

50. Overflow outlet

51. Fluid level

FIG. 3.

11. Photo-bioreactor

111. Photo-bioreactor flow inlet

112. Gas injector port

113. Photo-bioreactor flow outlet

114. Rib

115. Middle wall

116. Sidewall

FIG. 4.

15. LED panel light

151 frame

152. LED strip

153. LED bulb

154. light diffusion plate

155. light guide plate

156. reflective paper

157. back plate

We claim:

1. A photo-bioreactor panel comprising: side wall panels; edge fixtures joining the side wall panels a short distance apart forming an inner volume inside the panel, for growing a photosynthetic culture in an aqueous liquid, the photo-bioreactor panel comprising:
a fluid channel extending in the inner volume with an inlet and an outlet, the channel configured to provide a labyrinthine flow path from the inlet to the outlet and to contain the photosynthetic culture, wherein the channel is formed by a plurality of ribs between the inner surfaces of the side walls facing each other; and
a light emitting diode panel placed along a side wall of the photo-bioreactor.

2. The photo-bioreactor in claim 1, wherein the photo-bioreactor is oriented vertically.

3. The photo-bioreactor in claim 1, wherein at least some of the ribs are angled upward towards a top edge of the photo-bioreactor.

4. The photo-bioreactor in claim 4, wherein each angled rib is equidistant from another angled rib in a section.

5. The photo-bioreactor in claim 1, wherein the light emitting diode is planar to the channel.

6. Method of product preservation for a produced photosynthetic culture, comprising:
treating a packaged photosynthetic culture product with a first cycle of high pressure processing;
incubating the packaged product; and
treating the packaged product with a second cycle of high pressure processing.
